(19) United States
(12) Patent Application Publication
(10) Pub. No.: US 2004/0157270 A1
(43) Pub. Date: Aug. 12, 2004

(54) Protein measurement method in protein production plant by cell culture and apparatus thereof

(76) Inventors: Takeyuki Kondo, Hitachi (JP); Masaru Nanba, Hitachinaka (JP); Ryoichi Haga, Hitachi (JP)

Correspondence Address: MCDERMOTT, WILL & EMERY, 600 13th Street, N.W., Washington, DC, 20005-3096, US

(21) Appl. No.: 10/751,661

(22) Filed: Jan. 6, 2004

(30) Foreign Application Priority Data
Jan. 9, 2003 (JP) 2003-003322

(51) Int. Cl. G01N 33/53; G01N 33/567

(52) U.S. Cl. 435/7.2

(57) ABSTRACT

An automatic protein quantitative measurement apparatus having a reduced burden on an analysis operator, being free from bacterial contamination in each process device during collection of a sample liquid, having reduced measurement time and being capable of downsizing is provided. The automatic protein quantitative measurement apparatus has a sampling unit for automatically collecting a sample liquid online from at least one operation process of a cell culture step (wherein an antibody or other proteins are produced and secreted into the culture medium) and a separation step or a culture product purification step in a plant for culturing biological cells to produce a protein, a preprocessing unit for performing at least one of dilution and filtration of the sample liquid to adjust the liquid composition, a reaction unit having a cross-sectional area of 0.04 mm² or less and quantitatively measuring a protein contained in the adjusted liquid using an antigen-antibody reaction, a measurement unit for quantitatively measuring the protein according to the result of the reaction, a control unit for automatically controlling a series of operations of the above units constituting the apparatus, and a recording unit for recording the result of quantitative measurement.

### BACKGROUND OF THE INVENTION

(1) Field of the Invention

The present invention relates to a protein quantitative measurement method in a cell culture plant or the like, and an apparatus thereof.

(2) Description of the Related Art

A polymer protein pharmaceutical such as an antibody is produced by culturing biological cells in a culture tank. Then, a pharmaceutical as a product is separated from a culture solution, refined and dispatched. Since the quality and safety of the pharmaceutical should be assured, it is required to check whether the pharmaceutical has been actually treated normally not only in a biological cell culture step but also in a subsequent separation and refinement steps. In recent years, in particular, needs for quality assurance have grown since GMP (Good Manufacturing Practice for Pharmaceuticals) came into effect.

Hitherto, quantitative measurement of the polymer pharmaceutical has been performed in such a manner that an operator manually collects a culture solution or sample liquid from each operation step, and makes measurements by the ELISA (Enzyme-linked Immunosorbant Assay) method using micro-well plates. This assay operation has a large number of operation processes as described in “Immunology Illustrated (translated under supervision by Tomio Tada, issued by Nankodo, Jan. 1, 2000).

Furthermore, since quantitative measurements should be made manually for a large number of sample liquids collected at fixed time intervals from a plurality of production steps subsequent to a culture step, a burden on a measurement operator is enormous. Furthermore, since samples are collected from a culture tank manually, external bacteria and viruses may enter the culture tank to destruct culture cells.

Furthermore, in this method, an antigen-antibody reaction that is diffusion-controlled is made to proceed in a relatively large reaction field having a depth of about several millimeters, the measurement time required for obtaining an analysis result after taking a sample is therefore 3 hours or more, and thus the monitoring of culture states and the quality control of the product are not adequately performed. For performing the monitoring of culture states and high quality product control, time required for measurement should be reduced to about 20 minutes or less.

Methods enabling fast measurements and reducing a burden on operators may include an automatic measurement method using a high pressure liquid chromatography described in “High pressure Liquid Chromatography Handbook, second edition revised (edited by Japan Society for Analytical Chemistry, Kanto Division, issued by Maruzen).

In this method, separation/quantitative measurement is carried out using adsorption/desorption of a protein to a column, but a high pressure pump is required because a sample liquid should be fed to fine adsorption columns. Furthermore, pressure-resistant parts should be used in tubing, thus bringing about a problem such that an apparatus is prone to upsizing.

JP-A-2001-4628 discloses an immunoassay microchip in which a micro-channel reaction area and a micro-channel inflow area are formed on a glass substrate. The micro-channel reaction area is filled with solid particles, the antigen-antibody reaction is made to proceed on the solid particles, and an analysis is made according to photo-thermal conversion analysis.

Methods enabling fast measurements include a measurement method using a surface plasmon resonance (SPR) sensor as described in JP-A-2002-148258. In this method, an adsorption amount is measured using a shift of a resonance angle of a surface plasmon due to adsorption of a material on the surface of a metal thin film.

However, the resonance angle has very strong temperature dependency, thus there arises a problem such that strict temperature control within ±0.1° C. using a Peltier element or the like is required, and two sensors must be prepared to make a reference measurement if temperature control cannot be performed. Furthermore, because of susceptibility to impurities, a sensor should be calibrated for each measurement, and errors are significant although sensitivity is high. Furthermore, a rotation device of a laser source is required for scanning the resonance angle, and thus the apparatus configuration is complicated and expensive.

Problems associated with SPR, conventional ELISA method and HPLC (high pressure liquid chromatography) described above are listed as follows.

(1) SPR; strict temperature control and calibration for each measurement are required, errors are significant, and the apparatus is complicated and expensive.

(2) Conventional ELISA method; the analysis speed is low.

(3) HPLC; upsizing is caused, and the analysis speed is low.

### SUMMARY OF THE INVENTION

An object of the present invention is to provide a protein quantitative measurement apparatus and a measurement method enabling fast measurements and being free from contamination of each process device during collection of a sample liquid.

The present invention relates to a protein quantitative measurement apparatus comprising a sampling unit for taking a sample liquid online from a flow including a cell culture step (wherein an antibody or other proteins are produced and secreted into the culture medium), a separation step and a culture product purification step, a preprocessing unit for performing at least one of dilution and filtration of the sample liquid to adjust the liquid composition, a measurement unit for quantitatively measuring a protein contained in a liquid obtained by the adjustment by an antigen-antibody reaction, and a control unit for controlling a series of operations of apparatus with the above configuration, and a measurement method.

Other objects, features and advantages of the invention will become apparent from the following description of the embodiments of the invention taken in conjunction with the accompanying drawings.

### BRIEF DESCRIPTION OF THE DRAWINGS

FIG. 1 is a perspective view showing an outline of a microflow cell (microchip) in a protein automatic measurement apparatus according to the present invention.

FIG. 2 is a block diagram of one Example of a system of the protein automatic measurement apparatus according to the present invention.

FIG. 3 is a block diagram illustrating a specific configuration of a measurement unit that is used in the system of the present invention.

FIG. 4 is a block diagram illustrating means for switching between LDH measurement and antibody measurement that is used in the system of the present invention.

FIG. 5 is a block diagram illustrating protein quantitative measurement method by fluorometry or phosphorometry that is used in the system of the present invention.

FIG. 6 is a graph showing comparison of analysis results from the method of the present invention and the conventional ELISA method.

### PREFERRED EMBODIMENT OF THE INVENTION

A protein automatic measurement apparatus according to the present invention is comprised of a sampling unit for taking a sample liquid online at least one time from a flow including a cell culture step (wherein an antibody and other proteins are produced and secreted into the culture medium), a separation step subsequent to said cell culture and a culture product purification step in a plant for producing biological products such as protein, a preprocessing unit for performing at least one of dilution and filtration of the sample liquid to adjust the liquid composition so that the sample liquid can be passed through a microflow cell (microchip), a microflow cell (microchip) for making a protein contained in the adjusted liquid undergo a reaction with an enzyme, a measurement unit for quantitatively measuring the protein according to a result obtained from the reaction, a control unit for automatically controlling a series of operations of the above units constituting the apparatus, and a recording unit for recording a quantitative measurement result.

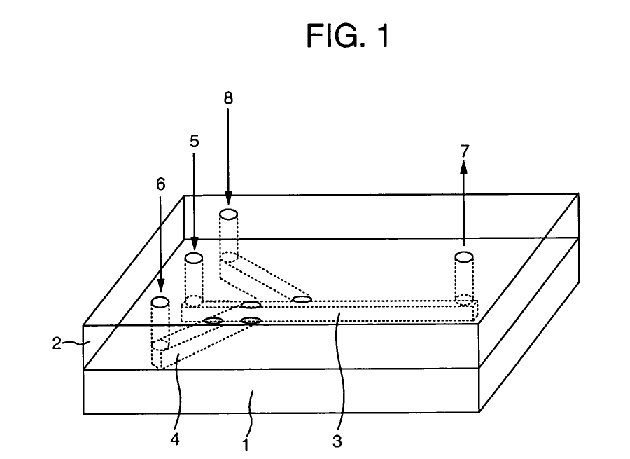

In the present invention, a micro-channel reaction apparatus described in JP-A-2001-4628 can be used as a microflow cell (microchip) for making a protein undergo a reaction with an enzyme to develop a color. Furthermore, the microflow cell (microchip) shown in FIG. 1 can be used.

Use of the microflow cell (microchip) can reduce reaction time considerably compared with use of liquid chromatography. The cross-sectional area of a reaction area of the microflow cell (microchip) should be 0.04 mm² or less. If the cross-sectional area is larger, the reaction time is prolonged and the reliability of an online monitor in a culture plant or the like is compromised.

As shown schematically in FIG. 1, the microflow cell (microchip) for use in the present invention is comprised of upper and lower two substrates 1 and 2. Micro-channels 3, 4 are formed on the lower substrate, and a sample and a reagent are fed through tubes 5, 6. The sample that has undergone a predetermined reaction is fed through tubes 7, 8 to an optical detection unit.

This microflow cell (microchip) is disposable, so that it is discarded after each reaction, and replaced with a new flow cell (microchip) when a sample is changed. The cross-sectional area of the micro-channel should be 0.04 mm² or less in relation to a reaction speed.

Thus, the sample must be passed through a very narrow flow path reaction unit, and it is therefore impossible to feed the reaction sample in its original form. Thus, it is required to dilute or filter the collected sample in advance so that the sample can pass through the narrow reaction path. In the present invention, both dilution and filtration may be performed as a matter of course.

In an analysis method described in JP-A-2001-4628, a photo-thermal conversion analysis using a gold colloid as a labelled antibody and using a thermal lens microscope is used, but because a light source for exciting the colloid is used, there arises a problem such that the apparatus is complicated and expensive. In the present invention, an enzyme and a protein are made to undergo a coloring reaction, and they are detected by an optical system, thus making it possible to extremely simplify the apparatus configuration and reduce a cost.

In the present invention, a protein-enzyme of interest and its substrate solution are made to contact each other while making them to flow at almost a constant rate in the microflow cell (microchip), whereby they are converted into a coloring pigment. Then, a quantitative measurement is made with an absorbance at a specified wavelength or a fluorescent coloring level. This process is used for detection of LDH (lactate dehydrogenase) and the like.

A primary antibody having a selective affinity for a product of interest may be fixed in the flow path of the microflow cell (microchip) in advance, the product of interest may be labelled with an enzyme using the principle of the Enzyme-Linked Immunosorbant Assay, and a quantitative measurement may be made using a coloring reaction with the enzyme. A microflow cell (microchip) for LDH detection and a microflow cell (micro chip) for antibody detection may be placed so that this antibody detection can be carried out in parallel with the detection of LDH described above.

The measurement unit according to the present invention includes means for feeding a plurality of reagents through a very small flow path, a displaceable very small flow path, a light source, an optical filter transmissive to only light of a specified wavelength of the lights emitted from the light source, an optical cell for pouring a colored solution by an enzymatic reaction, and a light-receiving element for measuring the intensity of light transmitting through the reagent in the optical cell.

Furthermore, liquid feed switching means is provided between the microflow cell (microchip) and the optical cell, so that liquid feed to the optical cell or a waste liquid tank can be selected.

According to the present invention, since the sample liquid from each step is collected online, there is no possibility that the external atmosphere and the culture liquid in a culture tank contact each other and that bacteria enters each apparatus of a cell culture step, a separation step and a purification step, and thus destruction of biological cells or degradation of the sample liquid in each step can be prevented. Furthermore, if the sample liquid is automatically collected instead of manual collection of the sample liquid by an operator, a burden on the operator can be considerably reduced.

Furthermore, since the collected sample liquid is diluted to adjust liquid conditions such as the salt concentration and pH, stability of the antigen-antibody reaction in the measurement unit can be improved, measurement errors can be reduced, and filtration can be eased.

Furthermore, by filtering the sample liquid, impurities can be prevented from being fed to the measurement unit, thus making it possible to protect a microflow cell (microchip) from blocking a micro-channel and so, improve measurement accuracy. Furthermore, because of the measurement method using the antigen-antibody reaction, it is not necessary to pass the sample liquid through a fine column as in high pressure liquid chromatography (HPLC) that is a conventional method, and the necessity to use a high pressure pump and pressure resistance specifications can be eliminated, thus making it possible to achieve downsizing of apparatus and a reduction in cost.

According to another embodiment of the present invention, the protein automatic measurement apparatus is characterized in that a protein is quantitatively measured by making the antigen-antibody reaction proceed in a very small flow path having a longitudinal cross-sectional area of 0.04 mm² or less in the measurement unit. According to the present invention, the antigen-antibody reaction that is diffusion-controlled is made to proceed in a very small flow path having a longitudinal cross-sectional area of 0.04 mm² or less, and therefore diffusion time is reduced, thus making it possible to considerably reduce quantitative measurement time.

It takes 3 hours for analysis with a reaction field having a depth of about 2 mm in the conventional measurement method, but by reducing the size of reaction field to a 0.2 mm square or less that is equivalent to 1/10 of that of the conventional method, the time required for measurement can be reduced to 1/100, and hence desired time of 5 minutes or less, thus making it possible to perform monitoring of a culture state and high quality product control.

According to still another protein automatic measurement apparatus of the present invention, any one of fluorometry, absorptiometry and phosphorometry is used as means for detecting a protein. According to the present invention, since absorptiometry is used as detection means, the apparatus can be downsized. This is because optical measurements can be made with a configuration of small simple devices such as a light source, an optical filter and a light-receiving element.

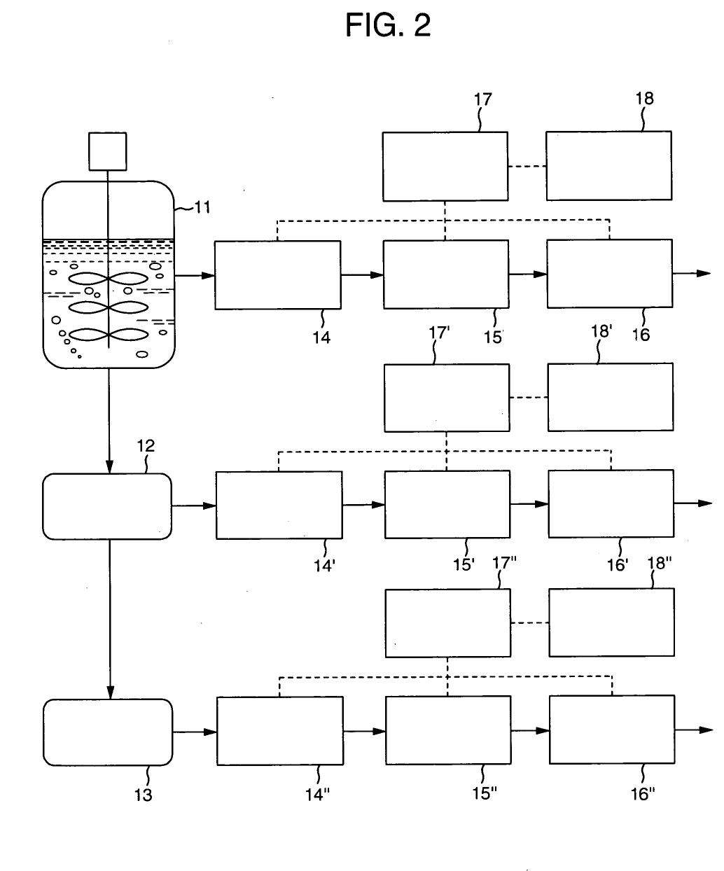

Embodiments of the present invention will be described in detail below with reference to the drawings. FIG. 2 is a system diagram of the protein automatic measurement apparatus. As shown in FIG. 2, the apparatus includes the following apparatus configuration.

(1) Online sampling units 14, 14′ and 14″ for automatically collecting a sample liquid from at least one operation step of a cell culture step (wherein an antibody or other proteins are produced and secreted into the culture medium) 11 and a separation process 12 or a culture product purification step 13 in a plant for culturing biological cells to produce a protein, or tubes and storage before and after the respective steps and the like.

Thus, a sample is collected from a flow including at least the above three steps. The sample may be further collected from other steps or before or after the steps as a matter of course.

(2) Preprocessing units 15, 15′ and 15″ for performing at least one of dilution and filtering of the sample liquid to adjust the liquid composition so that the sample liquid can be passed through the microflow cell (microchip).

(3) Measurement units 16, 16′ and 16″ for making a protein contained in the adjusted liquid emit light using the antigen-antibody reaction using the microflow cell (microchip) to quantitatively measure the protein. A light emitting signal can easily be detected using an optical system.

(4) Control units 17, 17′ and 17″ for automatically controlling a series of operations of apparatus with the above configuration.

(5) Recording units 18, 18′ and 18″ for recording a result of quantitative measurement.

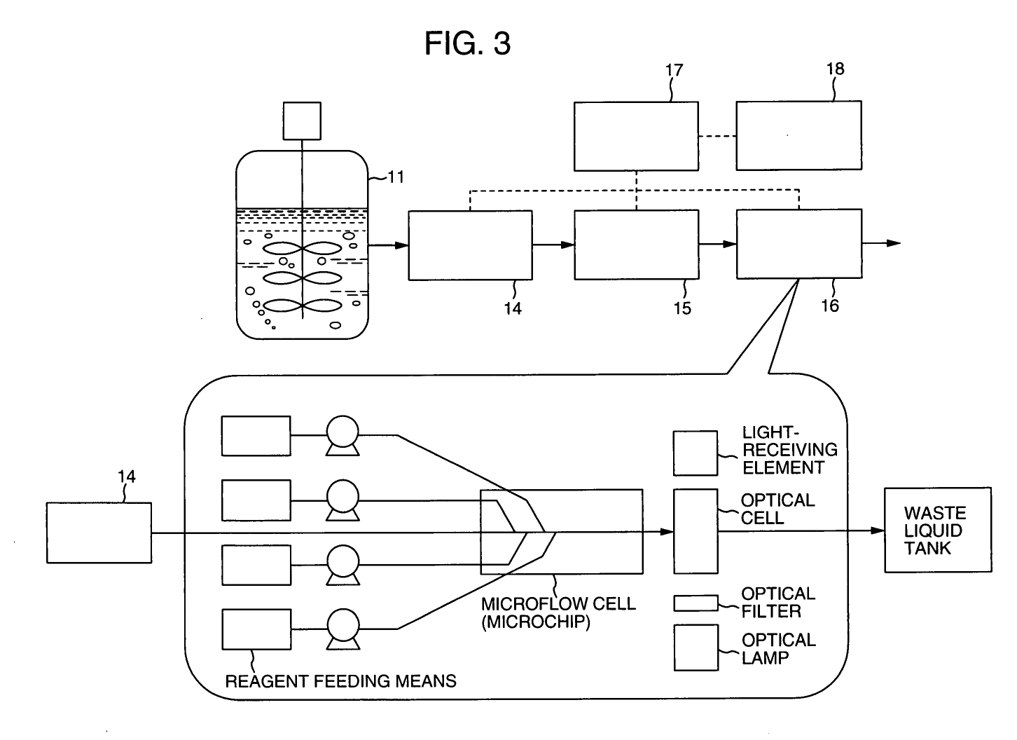

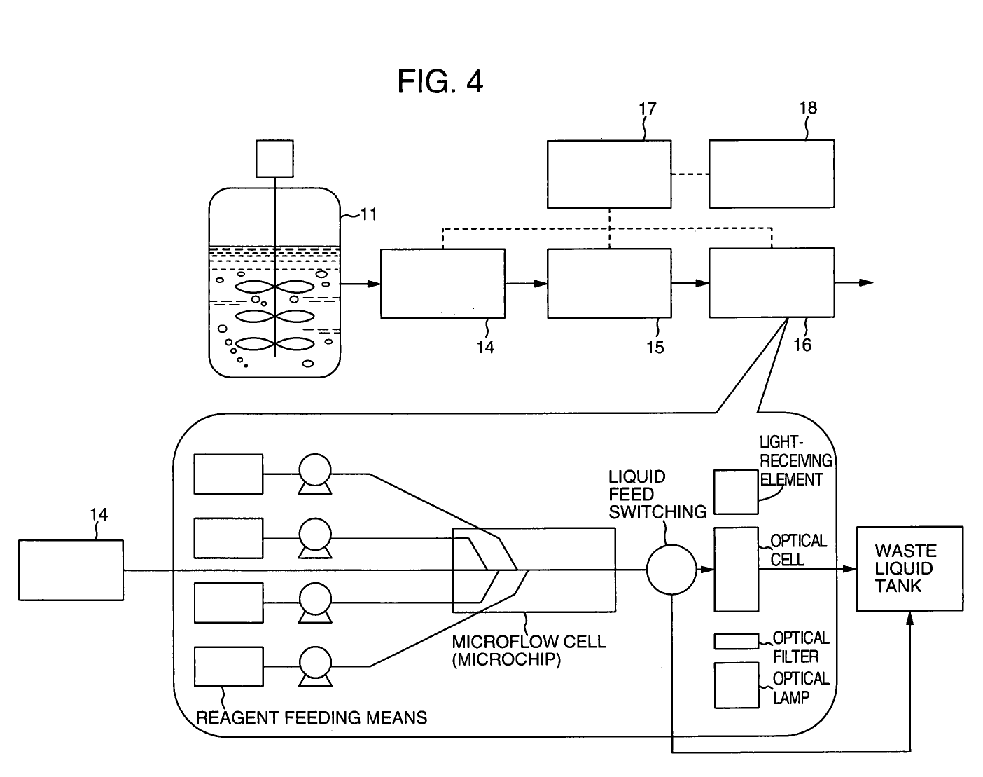

FIGS. 3 and 4 illustrate in detail a measurement system including the microflow cell (microchip). In FIGS. 3 and 4, like symbols denote like elements, and symbols same as those of FIG. 2 denote like elements.

In FIGS. 3 and 4, in the measurement unit 16, the sample collected in the sampling unit 14 is fed to the microflow cell (microchip), and a reagent(s) required for the sample are fed by a pump. A protein of interest in the sample are labelled with an enzyme after adsorbing a primary antibody which is fixed on polymer beads filled in the micro-channel of the microflow cell (microchip), and the enzyme reacts with a reagent(s) fed by a pump to develop a color. The sample is coupled to an optical cell together with the beads, and detected. The detected beads and sample are fed to a waste liquid tank.

FIG. 4 shows an apparatus configuration where a liquid containing the protein, of the sample liquid fed to the microflow cell (microchip), is directly fed to the waste liquid tank by liquid feed switching device such as a rotary valve after the microflow cell (microchip). Since the liquid containing the protein can be fed without being passed through the optical cell, deposition of the protein on the optical cell can be prevented, thus making it possible to reduce errors in the measurement result.

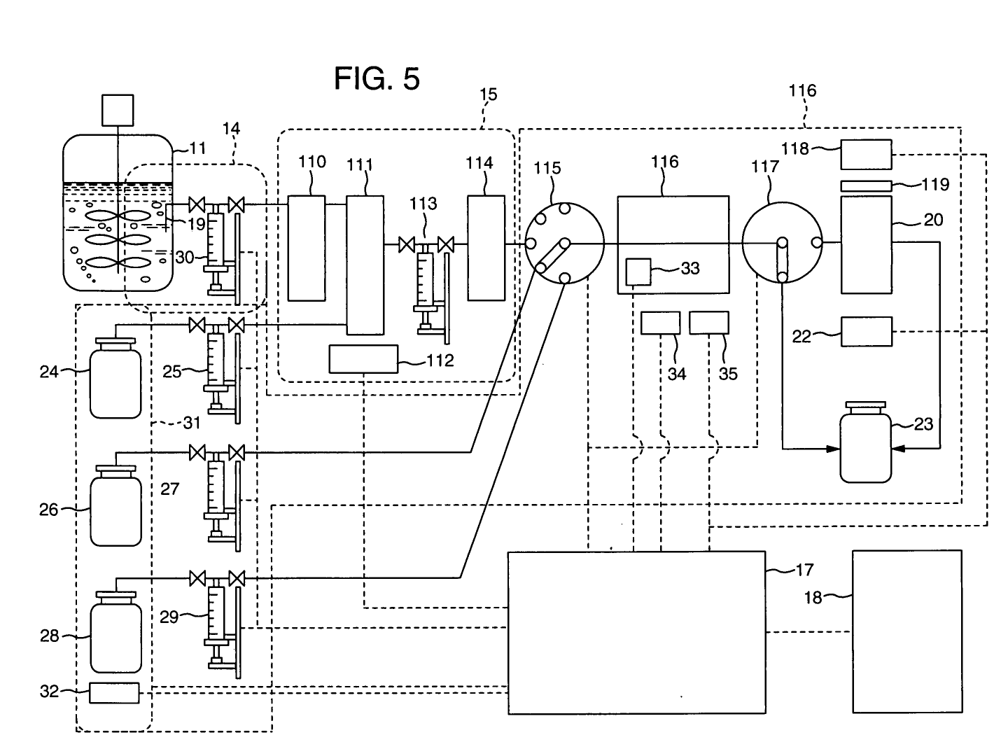

FIG. 5 shows one example of an apparatus configuration where a culture solution is collected from a culture tank, and a protein is quantitatively measured by the ELISA method. Operations beginning with the step of collecting the culture solution from the culture step and quantitatively measuring the protein by the ELISA method using the microflow cell (microchip), and ending with the step of recording the result will be described using FIG. 5.

Furthermore, proteins to be measured by the quantitative measurement apparatus include not only production proteins that are secreted into the culture solution as biological cells grow, but also enzyme proteins such as LDH that are flowed out from cells into the culture solution after biological cells die.

First, the culture solution is collected online from a sampling tube 19 connected to a culture tank 11 using a pump 30 so that the external atmosphere and the culture solution do not contact each other, and the collected culture solution is fed to the preprocessing unit 15. Since automatic collection is carried out online unlike a conventional manual collection, no bacteria enter from the external atmosphere. Thus, degradation of the sample liquid can be prevented, and a burden on the operator can be reduced. The pump is preferably a syringe pump in the sense that errors in the result of quantitative measurement are inhibited by reducing pulsation when the liquid is fed, but a piezo pump may be used.

The culture solution fed to the preprocessing unit 15 has impurities such as cell tissues, which are first roughly removed therefrom by a coarse filter 110, and is then fed to a dilution/mixing cell 111. At the same time, a specified amount of diluted liquid is fed from a diluted liquid tank 24 to the dilution/mixing cell 111 using a pump 25. The two liquids fed to the dilution/mixing cell 111 are mixed uniformly by a mixer 112, and then fed to precision filter 114 using a pump 27, and the sample liquid adjusted for measurement is fed to the measurement unit 16.

Since impurities such as cell tissues are filtered away in advance by the coarse filter 110, the reagent can easily be diluted in the dilution/mixing cell 111. Furthermore, by using a pH buffer solution for a diluting liquid, the pH of the sample liquid can be stabilized, thus making it possible to stabilize the subsequent antigen-antibody reaction in the measurement unit 16 to reduce measurement errors.

For the mixer 112, an ultrasonic vibration apparatus using a piezo element or a vibration mixer using a motor is desirably used in the sense that liquids are uniformly mixed in short time not in contact with the sample liquid, but a mixer using a magnetic stirrer may be used. Furthermore, since impurities that may cause clogging of the very small flow path of the measurement unit 16 in the subsequent stage are removed by the precision filter 114, the reagent flow in the very small flow path can be stabilized, thus making it possible to reduce measurement errors due to stabilization of the reaction.

In addition to the sample liquid adjusted in the preprocessing unit 15, reagents required for measurement by the ELISA method are fed to the measurement unit 16 from reagent tanks 26, 28 using pumps 27, 29. First, the adjusted sample liquid is made to flow through the microflow cell (microchip) (reaction tank) 116 in which a primary antibody for a protein (antigen) to be measured is immobilized in a solid phase in advance in a very small flow path having a sectional-area of 1 mm² or less, and coupled to the primary antibody. A secondary antibody liquid and an enzyme liquid are sequentially fed and coupled, and then a coloring reagent solution is made to flow.

The reagent solution contacts the coloring enzyme to develop a color in the measurement unit 16, and is fed to an optical cell 20 in the subsequent stage.

Light of a wavelength that is absorbed by a coloring reagent, of light produced in an optical lamp 118, is selected with an optical filter 119, and passed through the optical cell 20. The coloring absorbance of transmitting light is measured with a light-receiving element 22, and then the reagent solution is fed to a waste liquid tank 23.

Furthermore, since reagents other than the matrix solution contain proteins, measurement errors may occur due to adsorption of proteins on the cell surface if these reagents are fed to the optical cell 20. Therefore, reagents other than the matrix solution are selected and fed directly to the waste liquid tank by a liquid feed switching device 117.

For the liquid feed selection device 117, a rotary valve having a low dead volume may be used alone, or branched very fine flow path may be provided in the reaction tank 116 to provide a liquid feed selection function in the reaction tank 116. If the rotary valve having a low dead volume is used, contamination among reagents can be prevented, thus making it possible to improve measurement accuracy. Furthermore, if the branched very small flow path is used, it is possible to prevent degradation of the rotary valve caused by adsorption of the protein on the liquid contact surface.

One example of the reaction tank 116 is a micro reactor described in JP-A-2001-4628. It takes 3 hours for analysis with a reaction field having a depth of about 2 mm in the conventional measurement method, but by reducing the size of reaction field to a 0.02 mm square or less that is equivalent to 1/10 of that of the conventional method, the time required for measurement is reduced to 1/100, which means that it can be reduced to 5 minutes or less, thus making it possible to perform monitoring of a culture state and high quality product control.

The post-measurement sample liquid is fed to the waste liquid pool 23 to complete the analysis. Furthermore, the temperature of the reaction tank is measured by a temperature sensor 33, and temperature control is performed by a heater or Peltier element 34. Furthermore, because liquids in the dilution tank 24 and reagent tanks 26, 28 are easily degraded depending upon temperatures, temperature control is performed with the temperature sensor 33 and a cooler 31 so that those liquids can be stored at a temperature of 2 to 10° C.

Feed of the sample liquid and reagents by the pump, activation of the mixer of the dilution cell, operation of the liquid feed selection device, adjustment of temperature of the reaction tank, adjustment of temperature of a reagent repository, output of the measurement result and control of the optical system are all performed by a controller. The result of optical measurement is output to a recorder 8.

According to the Example described above, the sample liquid is collected online, and therefore there is no possibility that the external atmosphere and the culture liquid in a culture tank contact each other and that bacteria enters each apparatus of a cell culture step, a separation step and a purification step. Therefore, destruction of biological cells in the culture process, or degradation of the sample liquid in the separation/refinement process can be prevented. Furthermore, the sample liquid is automatically collected, a burden on an operator can be considerably reduced.

Furthermore, since the liquid conditions, such as the salt concentration and pH, of the collected sample liquid are adjusted in the preprocessing unit, stability of the antigen-antibody reaction can be improved, and measurement errors can be reduced. Furthermore, because of the measurement method using the antigen-antibody reaction, it is not necessary to pass the sample liquid through a fine column as in high pressure liquid chromatography (HPLC) that is a conventional method.

As a result, the necessity to use a high pressure pump and pressure resistance piping specifications can be eliminated, thus making it possible to achieve downsizing of apparatus and a reduction in cost.

Furthermore, since the antigen-antibody reaction that is diffusion-controlled is made to proceed in a very small flow path having a longitudinal cross-sectional area of 1 mm² or less, diffusion time is reduced to 1/9 of that of the conventional method, and thus quantitative measurement time can be reduced to 20 minutes or less. Furthermore, this makes it possible to perform monitoring of culture states and high quality product control.

Furthermore, since any of fluorometry, absorptiometry and phosphorometry is used as means for detecting a protein, optical measurements can be made with a combination of small simple devices such as a light source, an optical filter and a light-receiving element. Thus, downsizing of apparatus is possible.

According to the Example described above, the sample liquid is collected online, thus making it possible to prevent contact between the external atmosphere and the culture liquid. Consequently, entrance of bacteria into apparatus can be prevented, thus making it possible to prevent destruction of biological cells in the culture process, or degradation of the sample liquid in the separation/refinement process.

Because of automatic collection, a burden on an operator can be considerably reduced. Since the liquid conditions, such as the salt concentration and pH, of the collected sample liquid are adjusted in the preprocessing unit, stability of the antigen-antibody reaction can be improved, and measurement errors can be reduced.

Because of the measurement method using the antigen-antibody reaction, it is not necessary to pass the sample liquid through a fine column as in HPLC that is a conventional method, and therefore the necessity to use a high pressure pump and pressure resistance piping specifications can be eliminated, thus making it possible to achieve downsizing of apparatus and a reduction in cost.

Furthermore, since the antigen-antibody reaction that is diffusion-controlled is made to proceed in a very small flow path having a longitudinal cross-sectional area of 0.04 mm² or smaller, diffusion time is reduced to 1/100 of that of the conventional method, and thus quantitative measurement time can be reduced to 5 minutes or less. Furthermore, this makes it possible to perform monitoring of culture states and high quality product control.

Furthermore, since any of fluorometry, absorptiometry and phosphorometry is used as means for detecting a protein, optical measurements can be made with a combination of small simple devices such as a light source, an optical filter and a light-receiving element, thus making it possible to achieve downsizing of apparatus.

Since the sample liquid is collected online, there is no possibility that the external atmosphere and the sample liquid contact each other and that bacteria enters each apparatus of a cell culture step, a separation step and a purification step, thus making it possible to prevent destruction of biological cells in the culture process, or degradation of the sample liquid in the separation/refinement process.

Table 1 shows the time required for analysis when mouse-mouse hybridoma STK-1 capable of secreting and producing a DNA polymerase α antibody (IgG) was cultured in a 1L culture tank, and the concentration of antibody in the culture solution was measured by the method of the present invention and the conventional ELISA method.

In the measurement method of the present invention, chicken anti-mouse IgG was immobilized in a micro-flow path in advance, and a sample liquid containing IgG was made to flow therethrough at a rate of 50 μL/minute, followed by cleaning with a bovine serum albumin solution. Then, biotin-labelled horse anti-mouse IgG was made to flow therethrough, the bovine serum albumin solution was made to flow again, and then avidin-labelled alkali phosphatase was made to flow.

The bovine serum albumin solution was made to flow for cleaning; then p-nitrophenyl phosphate was made to flow; and the coloring absorbance was measured at 405 nm. Furthermore, in the conventional ELISA method, similarly a culture solution containing mouse IgG was injected into a well plate having chicken anti-mouse IgG adsorbed on the bottom in advance; the plate was cleaned; then the plate was cleaned with a bovine serum albumin solution; and biotin-labelled horse anti-mouse IgG was made to flow therethrough. Then, the bovine serum albumin solution was made to flow; then avidin-labelled alkali phosphatase was made to flow; the bovine serum albumin solution was made to flow therethrough for cleaning; then p-nitrophenyl phosphate was made to flow; and the coloring absorbance was measured at 405 nm.

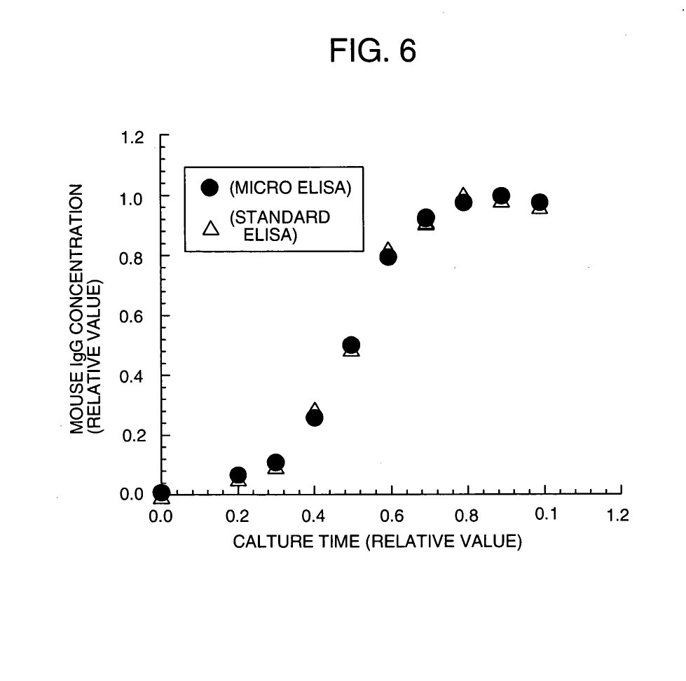

As a result, the conventional ELISA method required several hours for analysis, while in the analysis method of the present invention, a measurement could be made in about 5 minutes, and the amount of sample liquid could be reduced to 1/10 or less. Comparison of measurements results of both methods in FIG. 6 shows that the measurement performance obtained by the measurement method of the present invention are almost equivalent to those obtained by the conventional ELISA method.

TABLE 1

| | | Micro ELISA method | | Standard ELISA method | |
|---|---|---|---|---|---|
| Analysis flow | | Required time | Liquid amount | Required time | Liquid amount |
| 1) | Target protein (IgG) | 1 minute | 50 μL | 60 minutes | 50 μL |
| | coupling | | | | |
| | Plate cleaning | 1 minute | 50 μL | 5 minutes | 1200 μL |
| 2) | Secondary antibody | 1 minute | 50 μL | 30 minutes | 50 μL |
| | coupling | | | | |
| | Plate cleaning | 1 minute | 50 μL | 5 minutes | 1200 μL |
| 3) | Enzyme labeling | 1 minute | 50 μL | 30 minutes | 50 μL |
| | Plate cleaning | 1 minute | 50 μL | 5 minutes | 1200 μL |
| 4) | Coloring enzyme reaction | 0.5 minute | 25 μL | 3 minutes | 100 μL |
| | start | | | | |
| | Measurement of coloring | | | | |
| | absorbance (measurement | | | | |
| | wavelength 405 nm) | | | | |
| | Total | 6.5 minutes | 325 μL | 138 minutes | 3850 μL |

*Liquid flow rate: 50 μL/minute

According to the present invention, a quantitative measurement of a protein can be made in short time with a simple apparatus configuration, and contamination of a sample can be prevented, thus making it possible to perform highly reliable analysis.

It should be further understood by those skilled in the art that although the foregoing description has been made on embodiments of the invention, the invention is not limited thereto and various changes and modifications may be made without departing from the spirit of the invention and the scope of the appended claims.

What is claimed is:

1. A protein measurement method in a protein production plant by cell culture, wherein a sample liquid is sampled online one or more times from a flow including a cell culture step (wherein an antibody or other proteins are produced and secreted into the culture medium), a separation step subsequent to said cell culture and a culture product purification step; at least one of dilution and filtration of said sample liquid is performed to adjust the liquid composition so that said sample liquid can be passed through a microflow cell (microchip), a protein contained in a liquid obtained by the adjustment is made to undergo a coloring reaction with an enzyme in the microchip, and the protein is quantitatively measured.

2. The protein measurement method according to claim 1, wherein the microflow cell (microchip) is disposable.

3. The protein measurement method according to claim 1, wherein the cross-sectional area of a reaction area of the microflow cell (microchip) is 0.04 mm² or less.

4. A protein measurement apparatus in a protein production plant by cell culture, comprising:
a sampling unit for taking a sample liquid online at least one time from a flow including a cell culture step (wherein an antibody or other proteins are produced and secreted into the culture medium), a separation step subsequent to said cell culture and a culture product purification step in a plant for producing biological cells to produce a protein;
a preprocessing unit for performing at least one of dilution and filtration of the sample liquid to adjust the liquid composition so that the sample liquid can be passed through a microflow cell (microchip);
a microflow cell (microchip) for making a protein contained in a liquid obtained by the adjustment undergo a coloring reaction with an enzyme;
a measurement unit for quantitatively measuring the protein using a reaction result;
a control unit for controlling a series of operations of apparatus with the above configuration; and
a recording unit for recording a quantitative measurement result.

5. The protein automatic measurement apparatus according to claim 4, wherein the microflow cell (microchip) is disposable.

6. The protein automatic measurement apparatus according to claim 5, wherein the cross-sectional area of a reaction area of the microflow cell (microchip) is 0.04 mm² or less.
